(19) United States
(12) Patent Application Publication
(10) Pub. No.: US 2023/0256242 A1
(43) Pub. Date: Aug. 17, 2023

(54) MULTI-MODE MICROCURRENT STIMULUS SYSTEM AND ASSOCIATED METHODS

(71) Applicant: Tama Research Corporation, Scottsdale, AZ (US)

(72) Inventor: Tina ABNOOSI, Scottsdale, AZ (US)

(73) Assignee: Tama Research Corporation, Scottsdale, AZ (US)

(21) Appl. No.: 18/122,129

(22) Filed: Mar. 16, 2023

Related U.S. Application Data
(63) Continuation of application No. PCT/US2021/050853, filed on Sep. 17, 2021.
(60) Provisional application No. 63/079,732, filed on Sep. 17, 2020.

(51) Int. Cl. A61N 1/32; A61H 35/00; A61H 1/00; A61N 1/36; A61M 35/00

(52) U.S. Cl. CPC A61N 1/328; A61H 35/006; A61H 1/005; A61N 1/36034; A61M 35/10; A61H 2201/105; A61N 1/0452

(57) ABSTRACT

Methods of applying microcurrent stimulation to a subject may include providing a mask supplying at a minimum moisture to a region to be treated, applying a first electrode connected to a first polarity of microcurrent stimulation circuitry to the mask, applying a second electrode connected to a second polarity of the microcurrent stimulation circuitry to the subject outside of the mask, and providing microcurrent to the first electrode and the second electrode from the microcurrent stimulation circuitry. The method may further include inserting feet of the subject into a liquid having a minimum conductivity of 5 mS/m and vibrating the liquid.

### CROSS-REFERENCE TO RELATED APPLICATIONS

This patent application is a bypass continuation of PCT Application No. PCT/US2021/050853, filed Sep. 17, 2021, which claims the benefit of and priority to U.S. Provisional Patent Application No. 63/079,732, titled “MULTI-MODE MICROCURRENT STIMULUS SYSTEM AND ASSOCIATED METHODS,” filed on Sep. 17, 2020, each of which is herein incorporated by reference in its entirety.

### BACKGROUND

A microcurrent stimulation system with a power supply, two or more electrodes electronically coupled to the output drive circuit, a microcontroller configured to generate an electromagnetic waveform, an impedance measurement module configured to measure electrical impedance of one or more biological tissues between the two or more electrodes is disclosed in U.S. Pat. No. 8,731,657, which is hereby incorporated by reference for all purposes.

### SUMMARY

One or more embodiments are directed to providing a method of applying microcurrent stimulation to a subject. The method includes applying a first electrode connected to a first polarity of a microcurrent stimulation circuitry to a first side of the subject, applying a second electrode connected to a second polarity of the microcurrent stimulation circuitry to a second side of the subject, inserting feet of the subject into a liquid having a minimum conductivity of 5 mS/m, vibrating the liquid, and providing microcurrent to the first electrode and the second electrode from the microcurrent stimulation circuitry.

The liquid may extend above the feet of the subject. The liquid may cover ankles of the subject.

The first side may be a left side and the second side may be a right side. The first electrode may be applied to a left hand of the subject and the second electrode may be applied to a right hand of the subject.

The method may further include applying a third electrode connected to the first polarity of the microcurrent stimulation circuitry to the first side of the subject at an upper extremity of the subject, and applying a fourth electrode connected to the second polarity of the microcurrent stimulation circuitry to the second side of the subject to the upper extremity of the subject. The third electrode may be applied above a left shoulder of the subject and the fourth electrode may be applied above a right shoulder of the subject.

The first side and the second side may be a same side of the subject. The first electrode may be on an upper extremity of the subject and the second electrode is on a lower extremity of the subject.

The first electrode and the second electrode may be both on an upper extremity of the subject.

The method may further include applying a third electrode connected to the first polarity of the microcurrent stimulation circuitry to the first side of the subject at a lower extremity of the subject and applying a fourth electrode connected to the second polarity of the microcurrent stimulation circuitry to the second side of the subject to the lower extremity of the subject.

The method may further include applying a third electrode connected to the first polarity of the microcurrent stimulation circuitry to the first side of the subject at the upper extremity of the subject and applying a fourth electrode connected to the second polarity of the microcurrent stimulation circuitry to the second side of the subject to the upper extremity of the subject.

The first electrode and the second electrode may be both on a lower extremity of the subject.

A sheet mask may be provided between the first electrode and/or the second electrode and the subject.

Vibrating the liquid may be at frequency between 1 and 100 Hz.

The microcurrent may be 200 microamperes or less.

The method may further include determining a duration for providing the microcurrent based on a body mass index of the subject.

The liquid may be water.

The method may further include applying a first probe to the subject to activate the first electrode and a second probe to the subject to activate the second electrode.

The first electrode may be applied to under a right side of a chin of the subject and the second electrode may be applied to under a left side of the chin of the subject. The method may further include providing a chin brace under the chin of the subject.

One or more embodiments are directed to providing a method of applying microcurrent stimulation to a subject. The method may include applying a first electrode connected to a first polarity of microcurrent stimulation circuitry to a first side of the subject, applying a second electrode connected to a second polarity of the microcurrent stimulation circuitry to a second side of the subject, providing microcurrent to the first electrode and the second electrode from the microcurrent stimulation circuitry, and applying a first probe to the subject to activate the first electrode and a second probe to the subject to activate the second electrode.

One or more embodiments are directed to providing a method of applying microcurrent stimulation to a subject. The method may include providing a mask supplying at a minimum moisture to a region to be treated, applying a first electrode connected to a first polarity of microcurrent stimulation circuitry to the mask, applying a second electrode connected to a second polarity of the microcurrent stimulation circuitry to the subject outside of the mask, and providing microcurrent to the first electrode and the second electrode from the microcurrent stimulation circuitry.

The first electrode and the second electrode may be on a same side of the subject.

The first electrode and the second electrode may be centered on the subject.

The second electrode may be placed anywhere on the body.

The mask may include nutrients to be absorbed by the subject.

### BRIEF DESCRIPTION OF THE DRAWINGS

FIG. 1 illustrates an overview of a microcurrent stimulation system in accordance with an embodiment;

FIG. 2A illustrates an adjustable chair for use with the microcurrent stimulation system in accordance with an embodiment;

FIG. 2B illustrates an adjustable chair having circuitry for a microcurrent stimulation system integrated therein in accordance with an embodiment;

FIGS. 3A, 3B, and 4 illustrate additional configurations of the pad electrodes and/or the conductive gloves:

FIGS. 5-25, 34, 45, 46A-49, and 51-57 show various arrangements of the pad electrodes on the face, neck, and/or clavicle:

FIGS. 26, 27A, 27B, 28, 30, 31, 36, and 50 show arrangements in which pad electrodes are provided on the back of the legs and the feet, without using the foot bath or the conductive gloves;

FIG. 29 shows an arrangement of pad electrodes on the upper back using the conductive gloves;

FIGS. 32 and 33 show an arrangement of pad electrodes on the stomach;

FIG. 35 shows an arrangement of pad electrodes on the upper back without using the conductive gloves;

FIGS. 37 to 39 show arrangements of pad electrodes on the face and the front of the body including feet,

FIGS. 40 to 44 show arrangements of pad electrodes on the front and back of the body;

FIGS. 58-102 show various arrangements of the pad electrodes on the face and/or neck that includes use of a probe; and

FIGS. 103-106 show various probes in accordance with embodiments.

### DETAILED DESCRIPTION

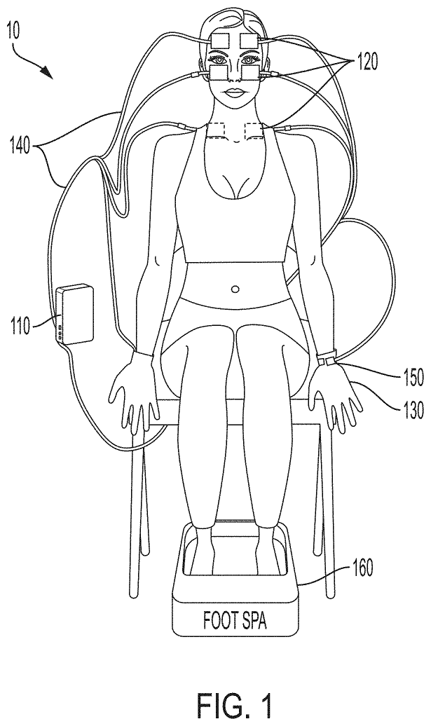

Instead of moving individual probes on specific regions, applicant has discovered that benefits of microcurrent may be realized using the same device on an individual as shown in FIG. 1. As shown in FIG. 1, which is a front view of an individual using the microcurrent stimulation system, the microcurrent stimulation system 10 includes microcurrent stimulation circuitry 110, e.g., BLUEONYX™ by TAMA Research, at least two pad electrodes 120 positioned on the individual, a pair of conductive gloves 130, e.g., gloves electrodes, on the individual, wires 140 that connect the pad electrodes 120 and the gloves 130 to the microcurrent stimulation circuitry 110, interface wires 150, e.g., a snap button, for the glove, and a foot bath 160 in which an individual's feet are inserted in a liquid having a minimum conductivity of 5 mS/m (milliSiemens per meter). For example, tap water conductivity is typically between 5-50 mS/m. Additionally, the liquid may vibrate at a frequency of 1-100 Hz, e.g., 13-27 Hz, by an ultrasonic transducer attached to the foot bath container Thus, microcurrent may be delivered throughout the body while the feet are electrically isolated from the microcurrent device. Alternatively, any manner of providing moisture to the bottom of the feet, e.g., a moist pad, a moist towel, a sheet mask, moist socks, and so forth, may be used for the foot bath 160.

Conductive gel may be provided between the pad electrodes 120 and/or the conductive gloves 130 and the individual. A total interface resistance may be between 0-1000 ohms for the system to operate properly. When applied throughout the body at appropriate electromagnetic waveforms, a stagnant lymphatic system may be released, the immune system may be boosted, and balance to the body's natural electromagnetic energy field may be restored while lifting, toning and nourishing the skin.

The microcurrent stimulation circuitry 110 includes or is connected to a power supply. In some implementations, the microcurrent stimulation circuitry 110 may include an electrical current generator, closed-loop feedback sensors, and intelligent circuitry to produce various pulse waveforms. The current may be in the range of 0-1000 microamperes. For example, lymphatic drainage may be achieved under 200 microamperes. The duration of application of the microcurrent is directly proportional to the body mass index (BMI) of the body.

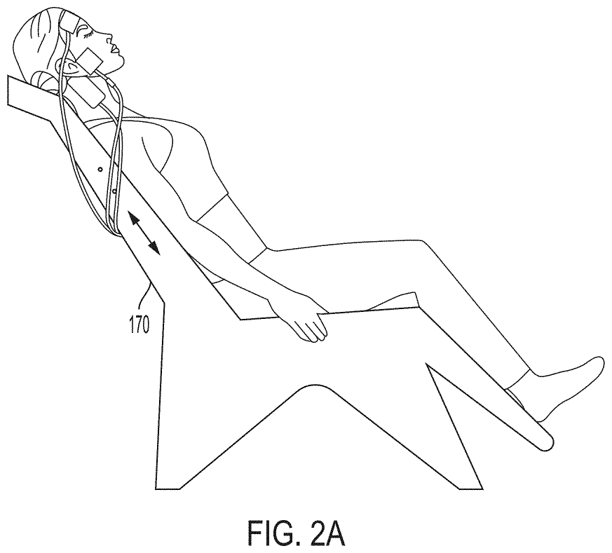

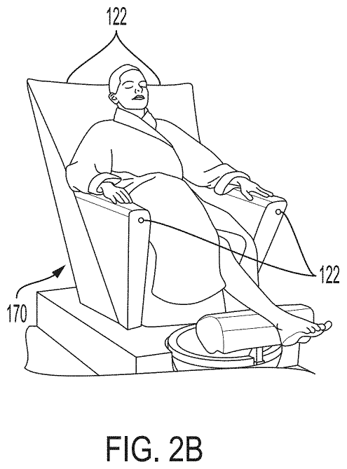

As shown in FIG. 1, at least two electrodes 120 may include a pair of electrodes above the eyes and a pair of electrodes below the eyes. As shown in FIG. 1, electrodes may also be provided on the collar bones and/or as shown in FIG. 2A, along the neck behind the ears. FIGS. 1-102 exemplify different embodiments of regarding placement of the pad electrodes 120. The body may be divided along a transverse plane, e.g., at the hips, into an upper extremity, e.g, above the hips and including the arms, and a lower extremity, e.g., below the hips. The body may also be divided along a vertical plane into left and right sides. Multiple sets of electrodes may be used at varying location along the body, as illustrated in the alternative configurations shown in the FIGS. 1-102.

The bath may be provided with sufficient liquid to cover pressure points of the lower extremity, e.g., may completely cover the feet and extend up to the ankles, while microcurrent is applied to the upper extremity. Opposite polarities may be applied to the left and right sides of the body. Electrodes may be provided at more than one location on either side of the body, e.g., to hands and anywhere else in the upper extremity, e.g., above the shoulders.

Throughout the drawing figures, the different colored connectors, e.g., dark gray and white or black and gray, indicate opposite polarities. In some drawing figures, the pad electrodes 120 are only provided on one side of the face/body to correct for asymmetry. Any of these arrangements may be used simultaneously on both sides the face/body. Sizes and shapes of the pad electrodes 120 may be adjusted in accordance with a size of the area being treated and concentration of the treatment to be delivered, as long as the pad electrodes are not in contact with each other. A narrow pad electrode may push more energy, i.e., more concentrated, in the area being stimulated. All arrangements herein may be used with or without the foot bath and with or without the conductive gloves.

As further shown in FIGS. 2A and 2B, the system 10 may also include an adjustable chair 170, as positioning an individual at an incline may further enhance the effect of the microcurrent simulation and better blood and lymphatic circulation. As shown in FIG. 2B, the microcurrent stimulation circuitry 110 may be integrated with the adjustable chair 170, which may include jacks 122 for connecting to the wires 140 for the pad electrodes 120. The adjustable chair may also fold the foot bath.

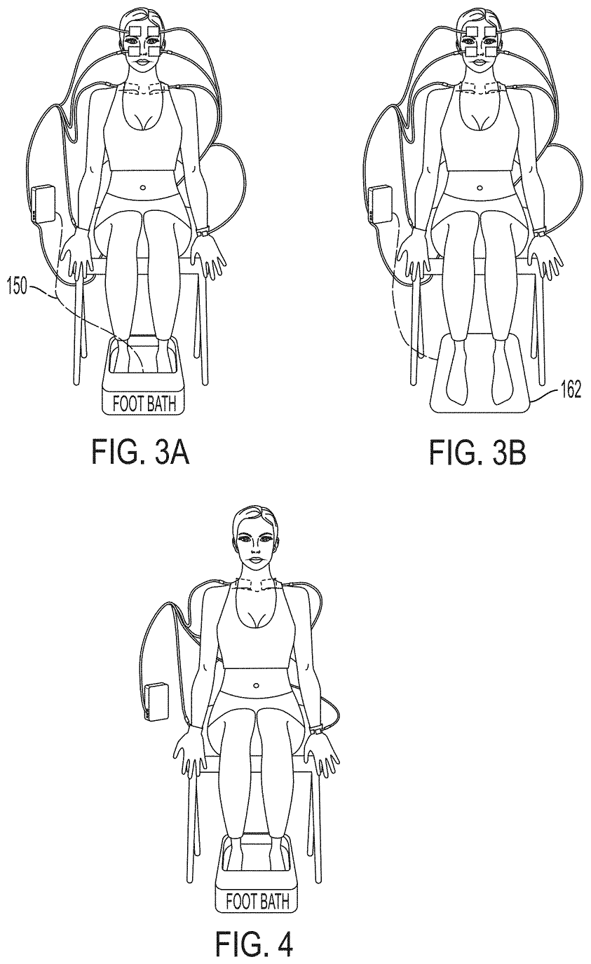

FIGS. 3A, 3B, and 4 illustrate additional configurations of the pad electrodes 120 and/or the conductive gloves 130. As shown in FIG. 3A, the interface wire 150 from the microcurrent stimulation circuitry 110 may be inserted into the foot bath 160. As shown in FIG. 3B, a moist pad 162 may replace the foot bath 160. As shown in FIG. 4, pad electrodes may not be placed on the face.

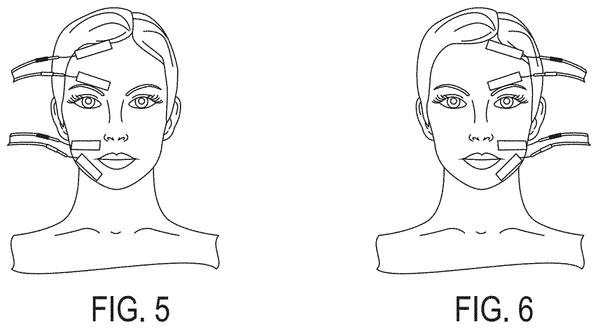

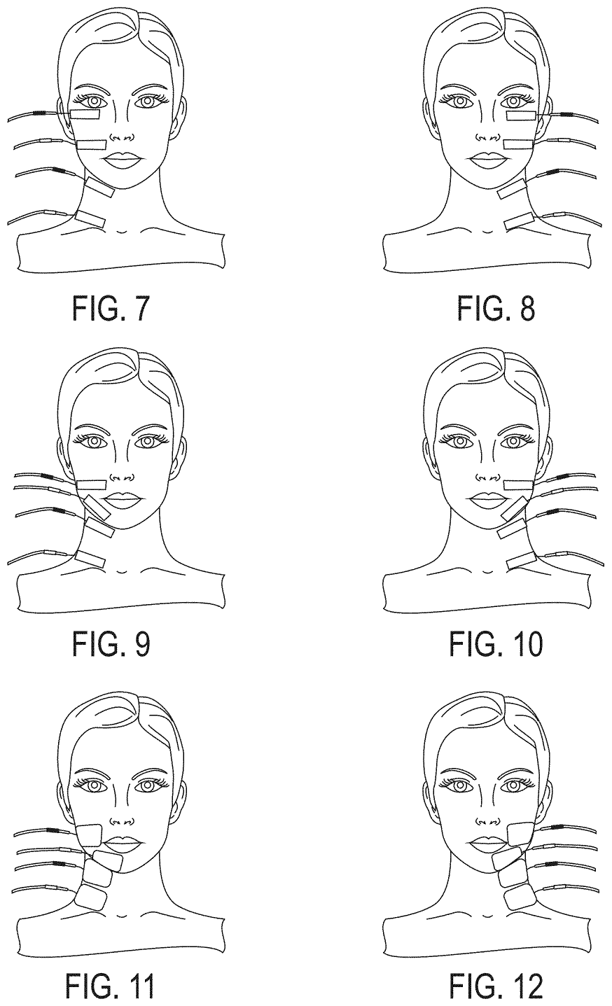

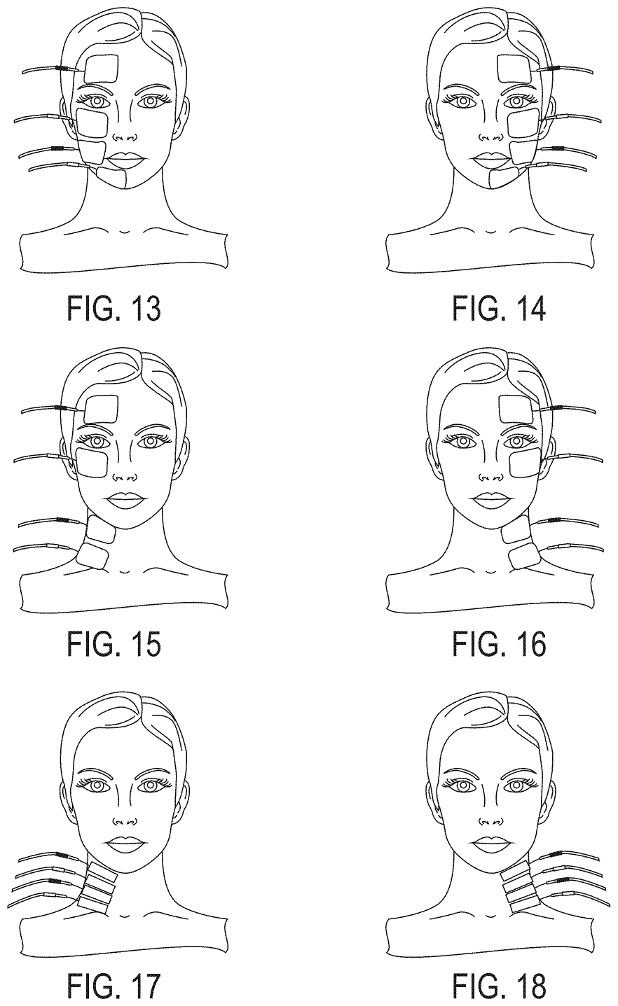

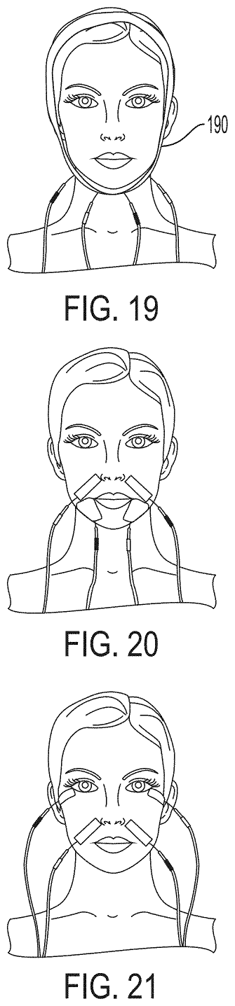

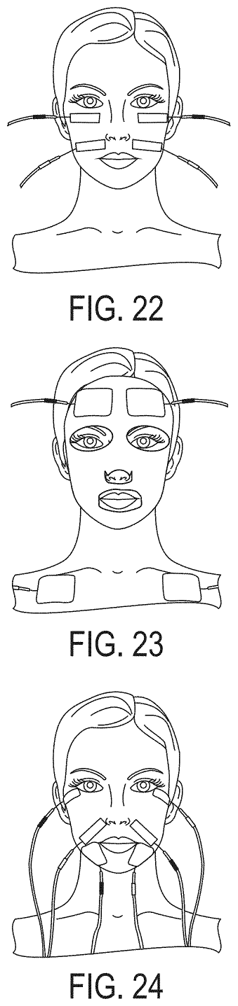

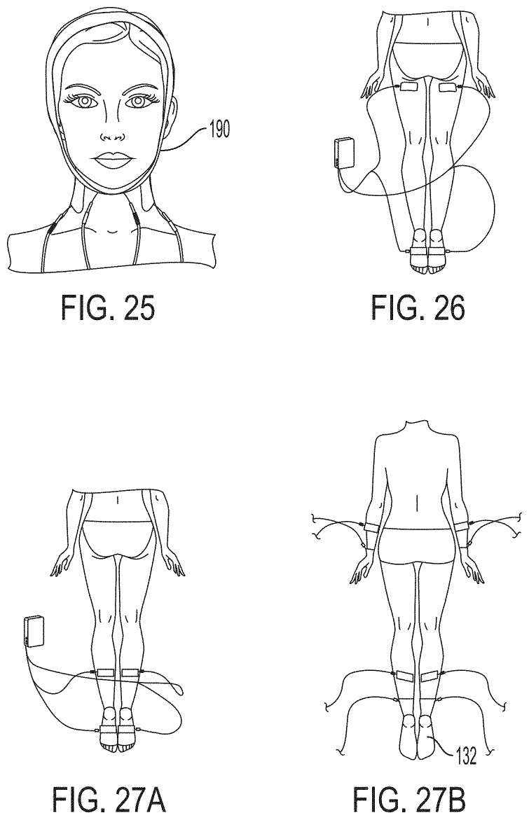

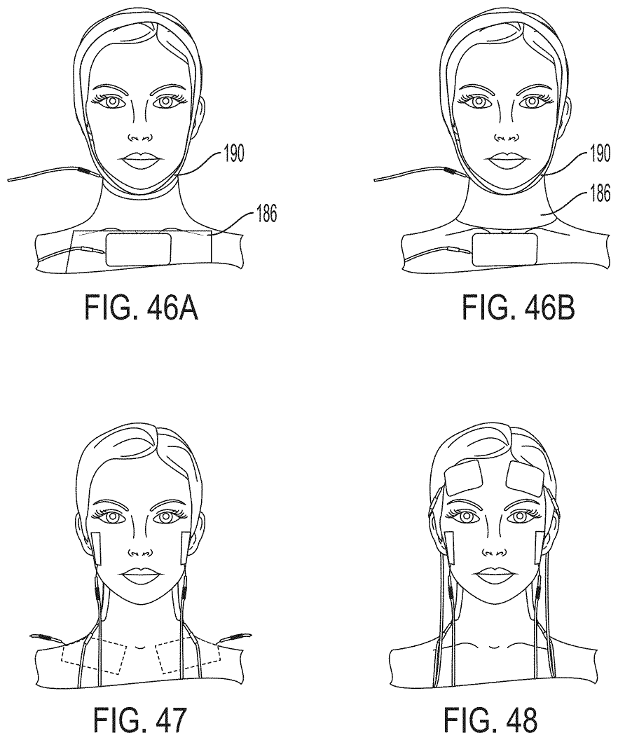

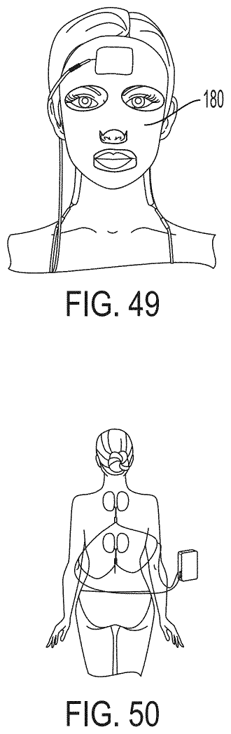

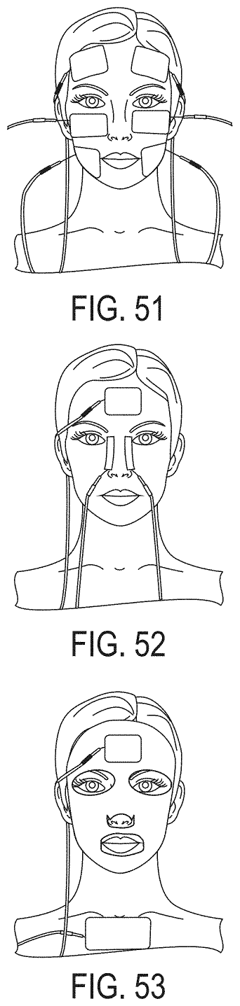

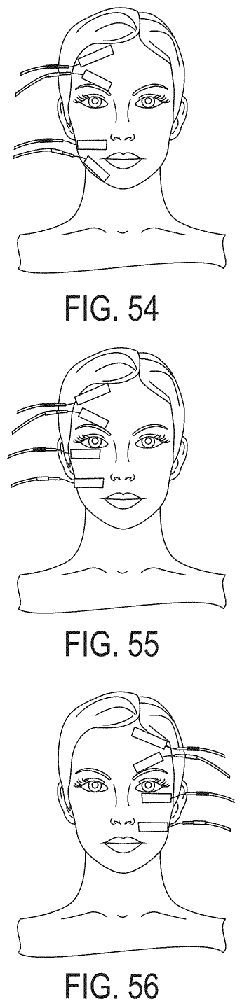

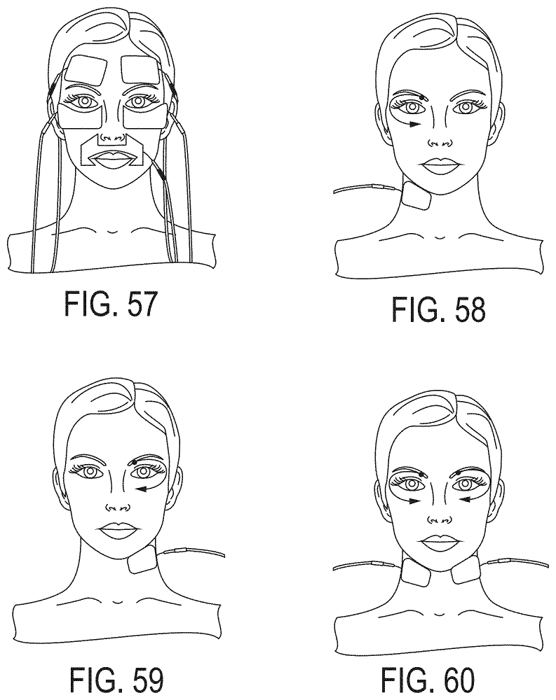

FIGS. 5-25, 34, 45, 46A-49, and 51-57 show various arrangements of the pad electrodes on the face, neck, and/or clavicle. As shown in FIGS. 5-18, 54, 55 and 56 the electrode pairs, e.g., two electrode pairs, may be on a same side. As shown in FIGS. 19-25, 34, 45, 47, 48, and 51, the electrode pairs may be symmetrically positioned. As shown in FIG. 24, there may be three electrode pairs, each pair having an electrode of different polarities. As shown in FIGS. 45, 48 and 51, there may be three electrode pairs, each electrode pair of electrodes has the same polarity, with a central pair having a different polarity than the upper and lower electrode pairs. As shown in FIGS. 49 and 52, a pair of electrodes having the same polarity and a single electrode, e.g., centered between the pair of electrodes, having an opposite polarity. As shown in FIG. 57, there may be two electrode pair, each electrode in the pair having a same polarity, while having opposite polarities from each other, and a single third electrode, e.g., centered between the electrode pairs in the traverse direction, and having an opposite polarity from the immediately adjacent electrode pair. As shown in FIGS. 46A, 46B, and 57, a single electrode pair, e.g., centered along a vertical direction, may be used.

As further shown in FIGS. 19, 25, 46A and 46B, a chin brace 190 that holds the heavy sternomastoid muscle in place so that a bond may be created between tissues during stimulation may be used.

As further shown in FIGS. 23, 49 and 53, the system 10 may also include a mask 180, e.g., a sheet mask, that provides moisture or a liquid to the area being treated as a means of delivering the microcurrent therapy to further enhance the effect of the microcurrent simulation and better lift and tone muscles, as well as infusing any product, e.g., nutrients, peptides, vitamins, and so forth in the mask. In particular, as shown in FIG. 23, a pad electrode 120a of a first polarity may be placed on the mask 180 on a left side and a pad electrode 120b of the second polarity may be placed on the left side, but not on the mask 180. A pad electrode 120a and a pad electrode 120b may be similarly placed on the right side. As shown in FIG. 49, a pad electrode 120a may be placed on the mask 180 at a center location and pad electrodes 120b may be placed off the mask on either side. As shown in FIG. 53, a pad electrode 120a may be placed on the mask 180 at a central location and a pad electrode 120b may be placed off the mask anywhere on the body, here shown on a center of the chest. These configurations may be used with or without the foot bath, with or without vibrating the liquid in the foot bath, and with or without the conductive gloves.

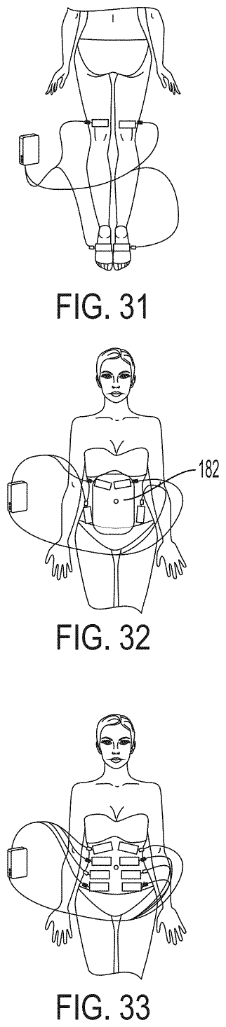

FIGS. 26, 27A, 27B, 28, 30, 31, and 36, show arrangements in which pad electrodes are provided on the back of the legs and the feet, without using the foot bath or the conductive gloves.

FIG. 27B shows an arrangement in which pad electrodes are provided on mid-calf and mid-forearm, while using the conductive gloves as well as conductive socks 162.

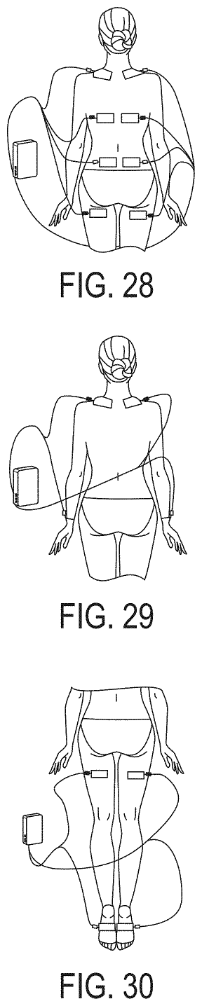

FIG. 28 shows an arrangement of pad electrodes on the upper, middle, and lower back, and upper back of the thighs.

FIG. 29 shows an arrangement of pad electrodes on the upper back using the conductive gloves.

FIGS. 32 and 33 show an arrangement of pad electrodes on the stomach. As shown in FIG. 32, a support 182 may be used to support stretched muscles in the abdomen, e.g., after pregnancy, to allow the region to heal and reduce scar tissue. As shown in FIG. 33, a plurality of electrodes spaced apart along a vertical direction of the area to be treated may be used.

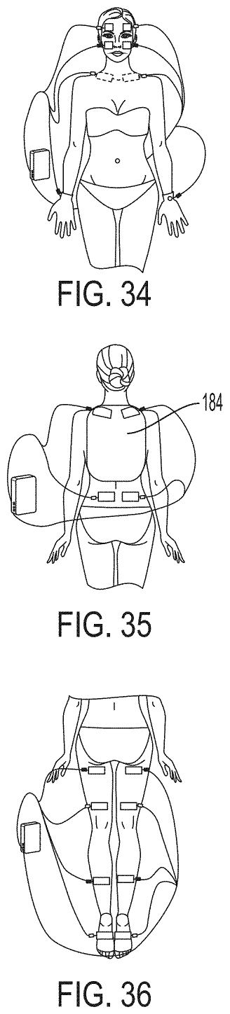

FIG. 34 shows an arrangement for dealing with removing inflammation and headache with no foot bath. As illustrated therein, the conductive gloves 130 may be used, along with electrode pad pairs above the eyes, below the eyes, and on the clavicle.

FIG. 35 shows an arrangement of pad electrodes on the upper and lower back, and including a support 184 extending form the upper back past the middle back used on the back to support the back muscles and on which the electrodes on the upper back are placed.

FIGS. 37-39 show arrangements for full body placement of pad electrodes FIG. 37 shows using two systems 10. In particular, as shown therein, electrode pads may be provided on one or both sides of the face, e.g., above and below the eyes, on the upper extremities, e.g., an armpit region and an inner elbow region, and on the lower extremities, e.g., an upper leg near the pelvic region, a front knee region, and an upper foot.

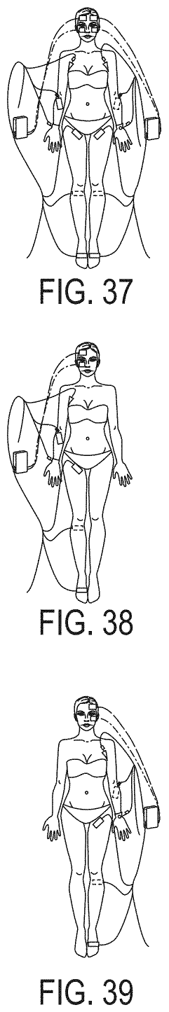

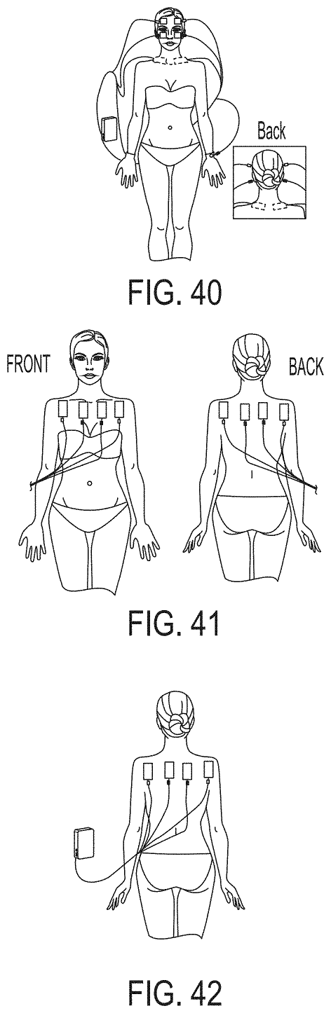

FIG. 40 shows an arrangement of pad electrodes on the face, clavicle, and upper back, used with the conductive gloves.

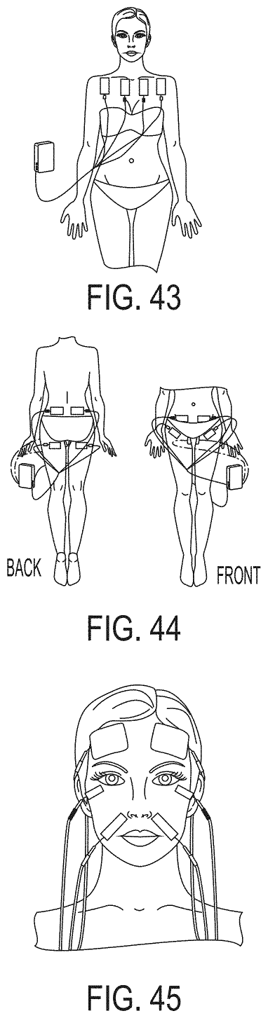

FIGS. 41-43 show arrangements of pad electrodes on the upper chest and upper back, that may be used either together or separately. As shown therein, the pad electrodes may include a plurality of pad electrodes, e.g., four, spaced along a traverse direction of the region being treated.

FIG. 44 shows an arrangement of pad electrodes adjacent the pelvic region and gluteal region, e.g., two pairs of electrodes on the front and on the back, with one pair of electrodes extending from the front to the back along inner thighs.

FIGS. 46A and 46B further includes the chin brace 190 that lifts and supports the neck and a support or sheet mask 186 on the upper chest or the neck. As further shown therein, a electrode under the chin may be provided and another electrode, e.g., in a center region of the upper chest, may be provided, either on or off the support or mask 186.

FIG. 50 shows an arrangement of pad electrodes on an upper and middle back.

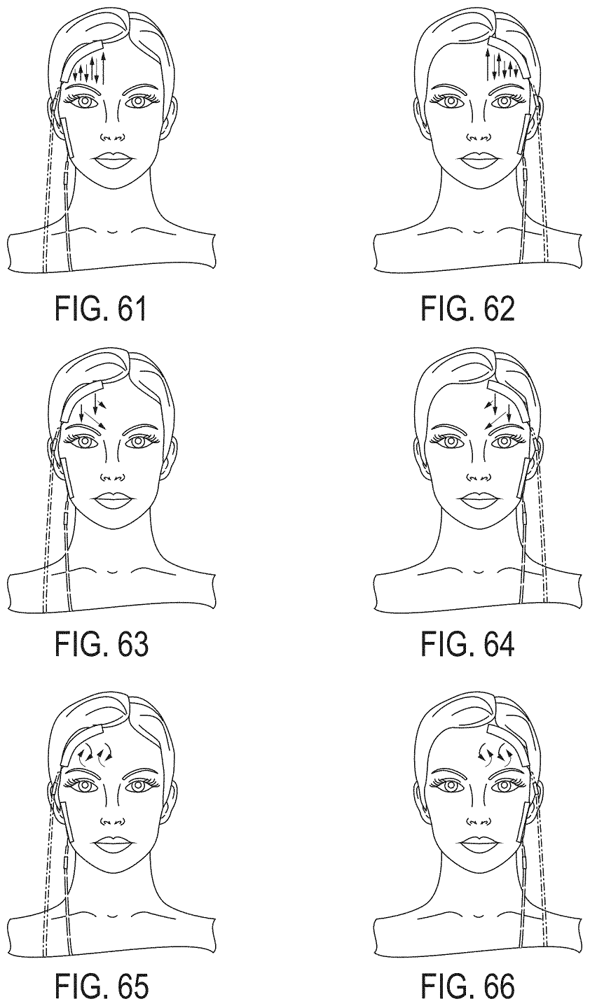

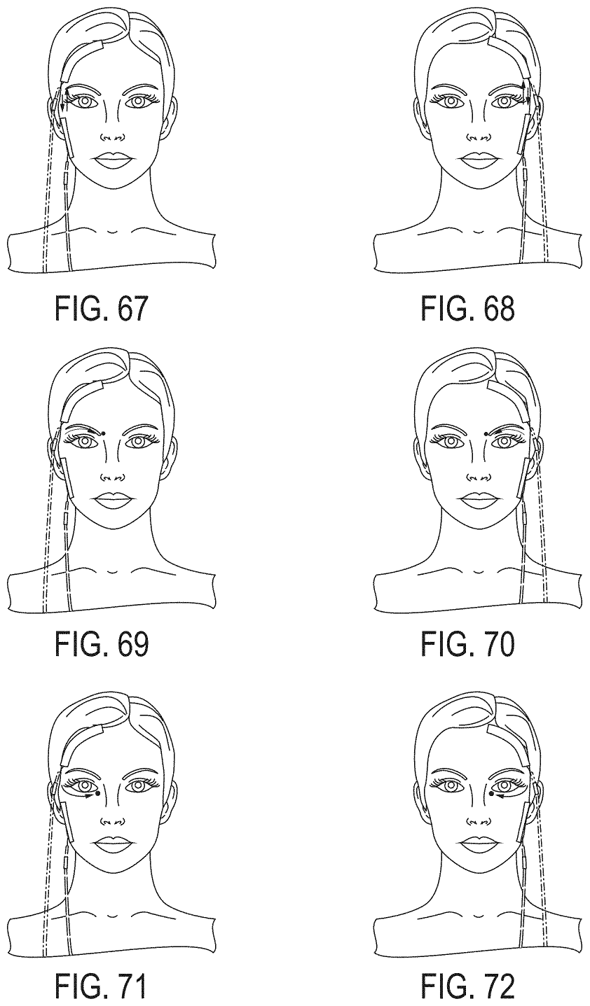

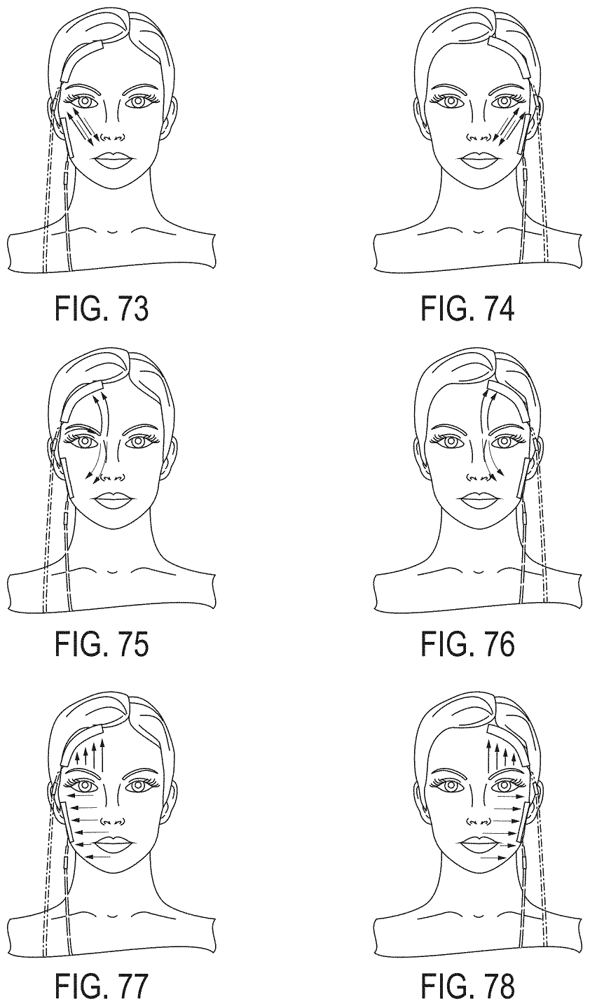

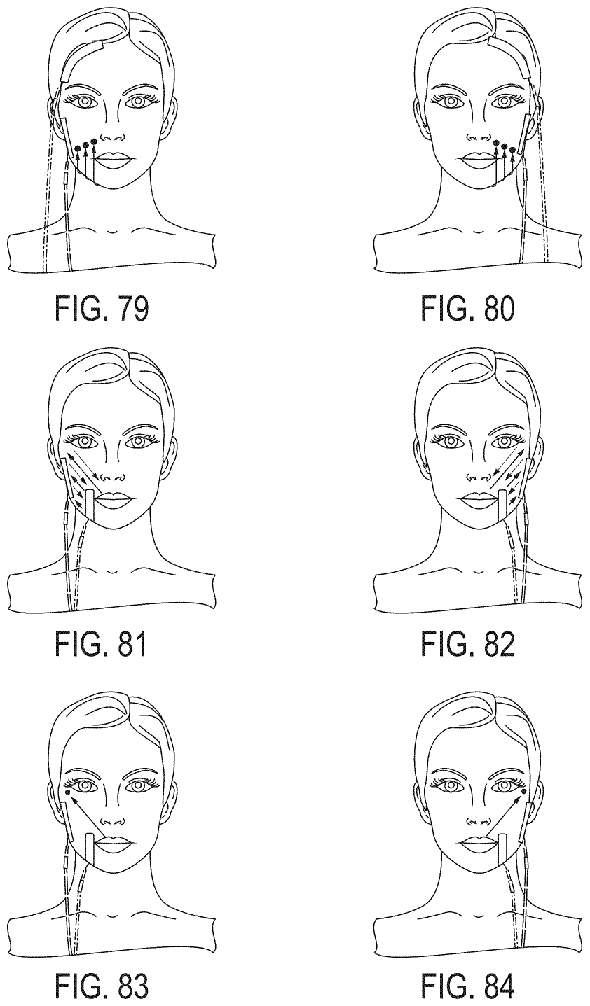

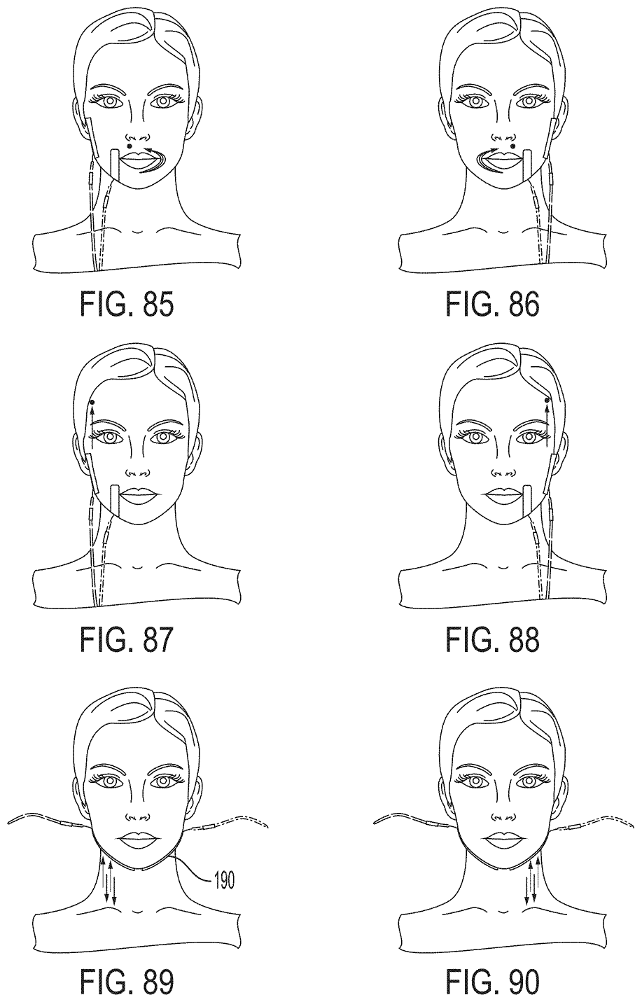

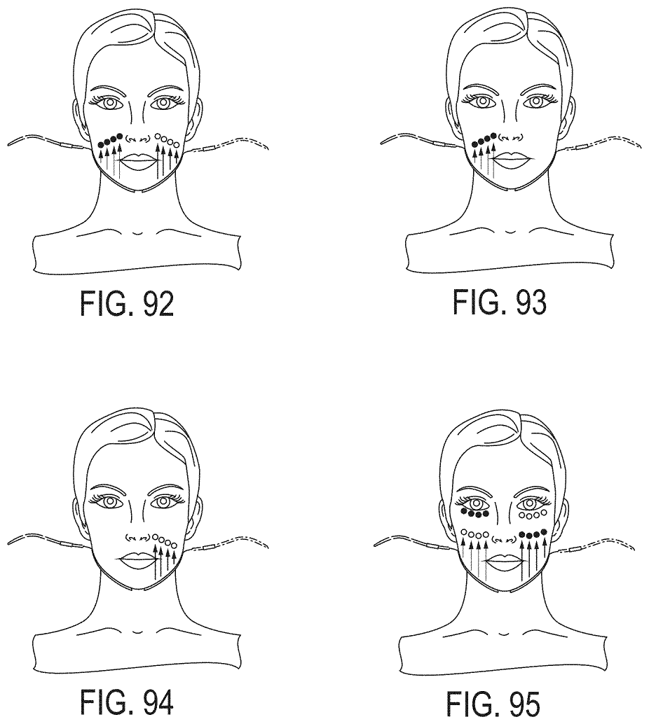

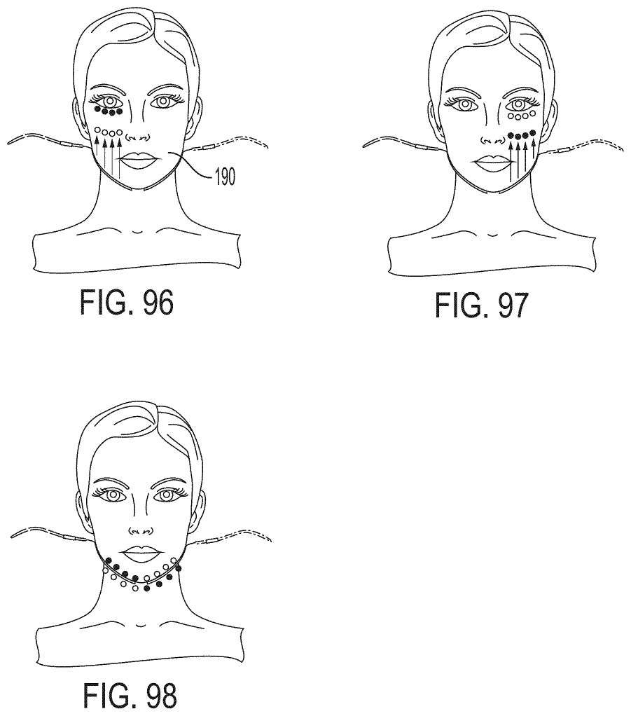

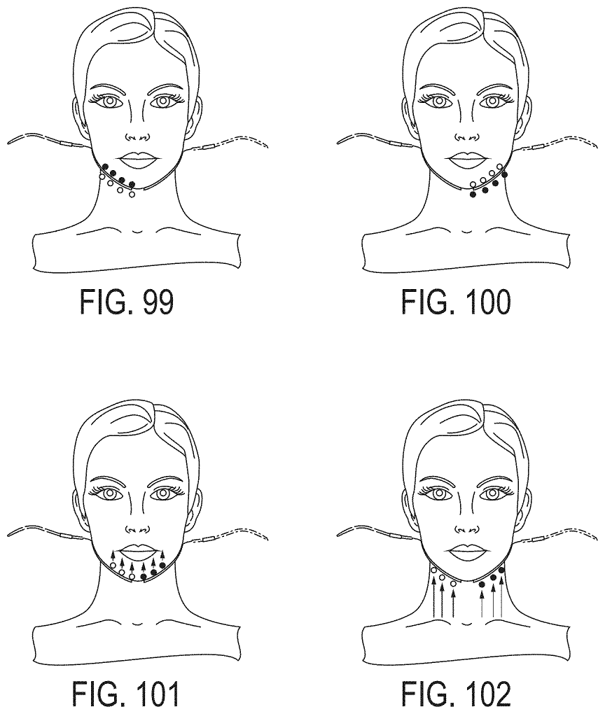

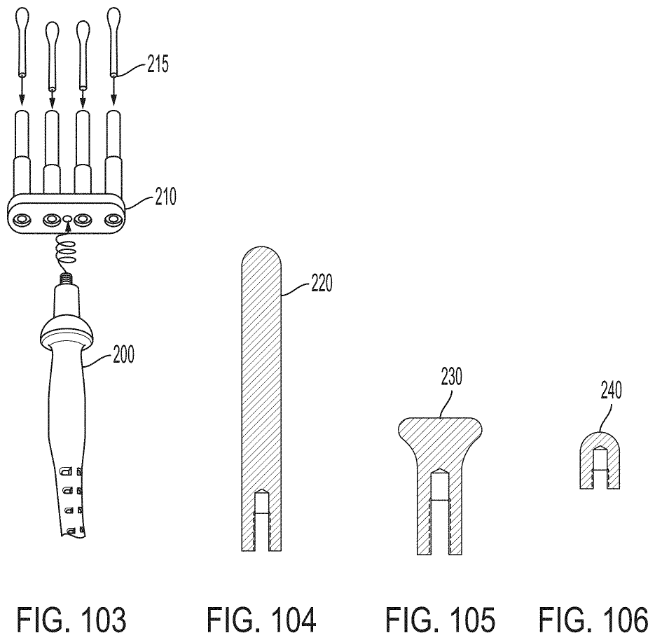

FIGS. 58-102 show various arrangements of the pad electrodes on the face and/or neck. These various arrangements may be used with or without the foot bath and with or without the conductive gloves. FIGS. 58-102 further show particular applications of probes that may be employed to further enhance the stimulation provided by the system 10. These probes may be the conductive gloves discussed above or may be a probe with various tips as shown in FIGS. 103-106. The arrows in FIGS. 58-102 indicate movement of the probes, while dots indicate stationary positions of the probes. The color of the probe application matches the pad electrode that is activated by the probe. When probe and pad are connected to wires with opposite polarity, they conduct to each other. The conduction path of electricity is lengthening and strengthening of the muscles by the movement of probe with respect to the position of the stationary pad.

As shown in FIG. 103, a probe 200, e.g., a TAMA™ probe produced by TAMA Research, may be connected to a first tip 210, e.g., a four-prong tip, into which moistened cotton swabs 215 are inserted. Alternatively, as shown in FIGS. 104-106, single prong tips, e.g., a long tip 220 with a curved end, a small tip 230 having a flat end, and a small tip 230 having a curved end, may be used with the probe 200. All of the tips may be made of e.g., stainless steel.

Generally accepted methodologies of microcurrent stimulation rely on currents at or below 1000 microamperes that mimic the body's own signals, but this disclosure is not intended to be limited to such current levels. Higher frequency pulses may penetrate deep into the skin tissue and reach the underlying muscle fibers. For example, the microcurrent stimulation circuitry 110 may apply a range of low frequencies, e.g., between 0.05 Hz and 5 KHz, in an alternating or interleaving manner to reach different depths in an individual, e.g., from the epidermis to the muscle. The microcurrent stimulation circuitry 110 may include a number of preset modes to cycle through to reach various depths. These modes may include interleaved, predetermined waveforms, i.e., having a preset frequency, amplitude, wave shape, etc., for a predetermined time period at over a predetermined total treatment time, minimizing user error and maximizing results.

Example embodiments have been disclosed herein, and although specific terms are employed, they are used and are to be interpreted in a generic and descriptive sense only and not for purpose of limitation. In some instances, as would be apparent to one of skill in the art as of the filing of the present application, features, characteristics, and/or elements described in connection with a particular embodiment may be used singly or in combination with features, characteristics, and/or elements described in connection with other embodiments unless otherwise indicated. Accordingly, various changes in form and details may be made without departing from the spirit and scope of the embodiments set forth in the claims.

What is claimed is:

1. A method of applying microcurrent stimulation to a subject, the method comprising:
applying a first electrode connected to a first polarity of a microcurrent stimulation circuitry to a first side of the subject;
applying a second electrode connected to a second polarity of the microcurrent stimulation circuitry to a second side of the subject;
inserting feet of the subject into a liquid having a minimum conductivity of 5 mS/m;
vibrating the liquid; and
providing microcurrent to the first electrode and the second electrode from the microcurrent stimulation circuitry.

2. The method of claim 1, wherein the liquid extends above the feet of the subject.

3. The method of claim 2, wherein the liquid covers ankles of the subject.

4. The method of claim 1, further comprising:
applying a third electrode connected to the first polarity of the microcurrent stimulation circuitry to the first side of the subject at an upper extremity of the subject; and
applying a fourth electrode connected to the second polarity of the microcurrent stimulation circuitry to the second side of the subject to the upper extremity of the subject.

5. The method of claim 1, wherein the first side and the second side are a same side of the subject.

6. The method of claim 5, wherein the first electrode and the second electrode are both on an upper extremity of the subject, and further comprising:
applying a third electrode connected to the first polarity of the microcurrent stimulation circuitry to the first side of the subject at a lower extremity of the subject; and
applying a fourth electrode connected to the second polarity of the microcurrent stimulation circuitry to the second side of the subject to the lower extremity of the subject.

7. The method of claim 5, wherein the first electrode and the second electrode are both on an upper extremity of the subject, and further comprising:
applying a third electrode connected to the first polarity of the microcurrent stimulation circuitry to the first side of the subject at the upper extremity of the subject; and
applying a fourth electrode connected to the second polarity of the microcurrent stimulation circuitry to the second side of the subject to the upper extremity of the subject.

8. The method of claim 1, wherein the first electrode and the second electrode are both on a lower extremity of the subject.

9. The method of claim 1, further comprising providing a sheet mask between the first electrode and/or the second electrode and the subject.

10. The method of claim 1, wherein vibrating the liquid includes vibrating the liquid at frequency between 1 and 100 Hz.

11. The method of claim 1, wherein the microcurrent is 200 microamperes or less.

12. The method of claim 1, further comprising determining a duration for providing the microcurrent based on a body mass index of the subject.

13. The method of claim 1, wherein the liquid is water.

14. The method of claim 1, further comprising applying a first probe to the subject to activate the first electrode and a second probe to the subject to activate the second electrode.

15. The method of claim 1, wherein the first electrode is applied to under a right side of a chin of the subject and the second electrode is applied to under a left side of the chin of the subject and further comprising providing a chin brace under the chin of the subject.

16. A method of applying microcurrent stimulation to a subject, the method comprising:
applying a first electrode connected to a first polarity of microcurrent stimulation circuitry to a first side of the subject;
applying a second electrode connected to a second polarity of the microcurrent stimulation circuitry to a second side of the subject;
providing microcurrent to the first electrode and the second electrode from the microcurrent stimulation circuitry; and
applying a first probe to the subject to activate the first electrode and a second probe to the subject to activate the second electrode.

17. A method of applying microcurrent stimulation to a subject, the method comprising:
providing a mask supplying at a minimum moisture to a region to be treated;
applying a first electrode connected to a first polarity of microcurrent stimulation circuitry to the mask;
applying a second electrode connected to a second polarity of the microcurrent stimulation circuitry to the subject outside of the mask; and
providing microcurrent to the first electrode and the second electrode from the microcurrent stimulation circuitry.

18. The method of claim 17, wherein the first electrode and the second electrode are on a same side of the subject.

19. The method of claim 17, wherein the first electrode and the second electrode are centered on the subject.

20. The method of claim 17, wherein the mask includes nutrients to be absorbed by the subject.
